(19) United States
(12) Patent Application Publication
(10) Pub. No.: US 2005/0154373 A1
(43) Pub. Date: Jul. 14, 2005

(54) Drain with occlusion removing structure

(76) Inventor: Harvey L. Deutsch, Los Angeles, CA (US)

Correspondence Address: SHELDON & MAK, INC, 225 SOUTH LAKE AVENUE, 9TH FLOOR, PASADENA, CA, 91101, US

(21) Appl. No.: 11/031,563

(22) Filed: Jan. 7, 2005

Related U.S. Application Data
(60) Provisional application No. 60/535,224, filed on Jan. 9, 2004.
(60) Provisional application No. 60/563,597, filed on Apr. 19, 2004.

(51) Int. Cl. A61M 31/00; A61M 1/00

(52) U.S. Cl. 604/540

(57) ABSTRACT

A drainage device (100) comprising means for drainage (102) and means for removing an occlusion (116, 136, 150). A method of unoccluding a means for drainage comprising a) providing the drainage device (100) according to the present invention; b) placing the drainage device into an area to be drained; c) allowing occluding material (134) to occlude the means for drainage (102); and d) removing the means for removing an occlusion (116, 136, 150) from the means for drainage (102), thereby unoccluding the means for drainage (102).

### CROSS-REFERENCE TO RELATED APPLICATIONS

The present Application claims the benefit of U.S. Provisional Patent Application 60/535,224 titled “Drain with Occlusion removing structure,” filed Jan. 9, 2004; and claims the benefit of U.S. Provisional Patent Application 60/563,597 titled “Drain with Occlusion removing structure,” filed Apr. 19, 2004; the contents of which are incorporated in this disclosure by reference in their entirety.

### BACKGROUND

There are a variety of medical drains, such as Penrose and Jackson-Pratt drains, used post-operatively to control blood and serosanguinous discharge from operative sites and wounds. Drains are typically used following major open abdominal operations, as well as during operations involving the back, spine, breast, head and hip. Drains help prevent the accumulation of hematomas and seromas in post-operative sites that lead to infections, abscesses, poor wound healing and wound dehiscence.

Medical drains generally have a distal end that is placed in the surgical site and a proximal end that is brought through the skin through a stab incision adjacent to the closed surgical incision. The proximal end of the drain is usually secured to the skin by one or more sutures.

Further, the proximal end of the drain is typically connected to a suction device, such as a compressible egg-shaped, container or bulb (a “hand grenade”). Connecting the suction device to the drain applies negative pressure through the drain and into the surgical site, encouraging the egress of fluid from the surgical site through the drain. The suction device is emptied of drainage fluid, and the amount of drainage is measured periodically, such as per nursing shift or per day. The drain is removed when the amount of drainage diminishes below a set amount during a specific time.

Disadvantageously, however, medical drains are prone to occlusion with inspissated bloody or serosanguinous drainage, allowing fluids to collect at the surgical site. When a drain occludes, it must be replaced, either by an open surgical procedure or by a minimally invasive surgical procedure, such as for example a procedure involving interventional radiology. Drain replacement procedures add significant extra expense and increase the risk of patient morbidity during the patient's post-operative course.

Therefore, there is a need for a new device or method that addresses the issue of occluded drains that involves less expense or less risk to the patient.

### SUMMARY

According to one embodiment of the present invention, there is provided a drainage device comprising means for drainage and means for removing an occlusion. According to another embodiment of the present invention, there is provided a method of unoccluding a means for drainage. The method comprises a) providing the drainage device; b) placing the drainage device into an area to be drained; c) allowing occluding material to occlude the means for drainage; and d) removing the means for removing an occlusion from the means for drainage, thereby unoccluding the means for drainage.

According to another embodiment of the present invention, there is provided a drainage device. The device comprises a drain comprising a proximal end, a distal end and a central lumen; and an occlusion removing structure within the central lumen comprising a proximal end and a distal end. The proximal end of the drain comprises an adaptor configured to mate with a suction device, the distal end of the drain comprises one or more than one aperture, the occlusion removing structure comprises one or more than one tubular structure comprising a wall, and the distal end of the occlusion removing structure comprises one or more than one aperture. In one embodiment, the drainage device further comprises a suction device mated with the adapter. In another embodiment, the occlusion removing structure comprises a plurality of co-axial tubular structures. In another embodiment, the number of the one or more than one aperture on the distal end of the drain is equal to the number of the one or more than one aperture on the distal end of the occlusion removing structure. In another embodiment, the proximal end of the occlusion removing structure comprises an appendage. In another embodiment, the appendage comprises an identification tag. In another embodiment, the proximal end of the drain comprises a cap. In another embodiment, the occlusion removing structure comprises an outer surface, the drain comprises an inner surface, and where the outer surface of the occlusion removing structure, or the inner surface of the drain, or both have a lubricious coating. In another embodiment, the drain further comprises one or more than one structure for securing the proximal end of the device. In another embodiment, the wall comprises a snare. In another embodiment, the occlusion removing structure is a first occlusion removing structure, and where the device further comprises a second occlusion removing structure comprising a proximal end, a distal end, an intermediate segment between the proximal end and the distal end, and an inflation balloon on the distal end.

According to another embodiment of the present invention, there is provided a method of unoccluding a drainage device. The method comprises a) providing the drainage device of according to the present invention; b) placing the distal end of the drain into an area to be drained; c) allowing occluding material to occlude the drain; and d) removing the occlusion removing structure from the drain, thereby unoccluding the drain. In one embodiment, the method further comprises attaching the drainage device to a suction device after placing the distal end of the drain. In another embodiment, the method further comprises reattaching the drainage device to the suction device after removing the occluding material removing structure. In another embodiment, the occlusion removing structure comprises an appendage, and removing the occlusion removing structure comprises applying proximal traction to the appendage, thereby translating the occlusion removing structure proximally. In another embodiment, the drain comprises a cap, and removing the occlusion removing structure comprises opening the cap to permit the occlusion removing structure to be removed from the drain. In another embodiment, the occlusion removing structure comprises a plurality of co-axial tubular structures, and removing the occlusion removing structure preferably comprises removing only one of the plurality of co-axial tubular structures. In another embodiment, the method further comprises infusing a fluid between each of the plurality of co-axial tubular structures to facilitate separation of the co-axial structures from each other. In another embodiment, the wall comprises a snare, and removing the occlusion removing structure comprises applying proximal traction to the proximal end of the snare causing the distal end of the occlusion removing structure to contract in a purse-string type fashion, thereby substantially or completely sealing the distal end of the occlusion removing structure.

According to another embodiment of the present invention, there is provided a drainage device. The device comprises a drain comprising a proximal end, a distal end and a central lumen, and an occlusion removing structure within the central lumen comprises a proximal end and a distal end. The proximal end of the drain comprises an adaptor configured to mate with a suction device, the distal end of the drain comprises one or more than one aperture, and the occlusion removing structure comprises a tubular mesh. In one embodiment, the proximal end of the occlusion removing structure comprises a plurality of loops. In another embodiment, the drainage device further comprises a suction device mated with the adapter. In another embodiment, the proximal end of the occlusion removing structure comprises an appendage. In another embodiment, the proximal end of the drain comprises a cap. In another embodiment, the drain further comprises one or more than one structure for securing the proximal end of the device. In another embodiment, the occlusion removing structure comprises a connector on the proximal end. In another embodiment, the device further comprises a joint between the adaptor and the connector, and further comprises a sealant for sealing the joint between the connector and the adaptor. In another embodiment, the occlusion removing structure is a first occlusion removing structure, and where the device further comprises a second occlusion removing structure comprising a proximal end, a distal end, an intermediate segment between the proximal end and the distal end, and an inflation balloon on the distal end.

According to another embodiment of the present invention, there is provided a method of unoccluding a drainage device. The method comprises a) providing the drainage device of claim 22; b) placing the distal end of the drain into an area to be drained; c) allowing occluding material to occlude the drain; and d) removing the occlusion removing structure from the drain, thereby unoccluding the drain. In one embodiment, the method further comprises attaching the drainage device to a suction device after placing the distal end of the drain. In another embodiment, the method further comprises reattaching the drainage device to the suction device after removing the occluding material removing structure. In another embodiment, the method where the occlusion removing structure comprises an appendage, and where removing the occlusion removing structure comprises applying proximal traction to the appendage, thereby translating the occlusion removing structure proximally. In another embodiment, the method where the drain comprises a cap, and where removing the occlusion removing structure comprises opening the cap to permit the occlusion removing structure to be removed from the drain. In another embodiment, the method where the proximal end of the occlusion removing structure comprises loops, and where removing the occlusion removing structure from the drain comprises pulling the proximal end of the occlusion removing structure into a sheath. In another embodiment, the method further comprises replacing the occlusion removing structure in the drain after unoccluding the drain. In another embodiment, replacing the occlusion removing structure in the drain after unoccluding the drain comprises advancing an introducer containing the replacement occlusion removing structure through the proximal end of the drain until the distal end of the replacement occlusion removing structure is placed at or near the distal end of the drain, and then removing the introducer leaving the replacement occlusion removing structure within the drain.

According to another embodiment of the present invention, there is provided a drainage device. The device comprises a drain comprising a proximal end, a distal end and a central lumen, and b) an occlusion removing structure within the central lumen comprises a proximal end, a distal end and intermediate segment between the proximal end and the distal end. The proximal end of the drain comprises an adaptor configured to mate with a suction device, the distal end of the drain comprises one or more than one aperture, and the occlusion removing structure comprises an inflation balloon on the distal end. In one embodiment, the drain further comprises a suction device mated with the adapter. In another embodiment, the proximal end of the drain comprises a cap. In another embodiment, the drain further comprises one or more than one structure for securing the proximal end of the device. In another embodiment, the drain further comprises a collapsible sleeve.

According to another embodiment of the present invention, there is provided a method of unoccluding a drainage device comprising a) providing the drainage device according to the present invention; b) placing the distal end of the drain into an area to be drained; c) allowing occluding material to occlude the drain; d) inflating the inflatable balloon; and e) removing the occlusion removing structure from the drain, thereby unoccluding the drain. In one embodiment, the method further comprises attaching the drainage device to a suction device after placing the distal end of the drain. In another embodiment, the method further comprises reattaching the drainage device to the suction device after removing the occluding material removing structure. In another embodiment, the drain comprises a cap, and removing the occlusion removing structure comprises opening the cap to permit the occlusion removing structure to be removed from the drain. In another embodiment, the method further comprises replacing the occlusion removing structure in the drain after unoccluding the drain. In another embodiment, the method further comprises replacing the occlusion removing structure in the drain after unoccluding the drain. In another embodiment, the drain further comprises a collapsible sleeve, and the collapsible sleeve is extended proximally as the occlusion removing structure is removed from the drain.

### FIGURES

These and other features, aspects and advantages of the present invention will become better understood with regard to the following description, appended claims, and accompanying figures where:

FIG. 1 is a partial, lateral perspective view of one embodiment of a drainage device according to the present invention comprising a drain with an occlusion removing structure, shown with a suction device attached;

FIG. 2 is a partial, cutaway, lateral perspective view of the embodiment of the device shown in FIG. 1;

FIG. 3 is a partial, cutaway, lateral perspective view of another embodiment of a drainage device shown in FIG. 1, with an alternate embodiment of the occlusion removing structure;

FIG. 4 is a cross-sectional view of the device shown in FIG. 3, taken along line 4-4;

FIG. 5 is a close-up view of the distal end of the occlusion removing structure of one embodiment of the drainage device shown in FIG. 1 comprising a snare with the snare open;

FIG. 6 is another close-up view of the distal end of the occlusion removing structure of one embodiment of the drainage device shown in FIG. 1 comprising a snare with the snare closed;

FIG. 7 is another partial, cutaway, lateral perspective view of the embodiment of the device shown in FIG. 1 showing an occlusion being removed from the device according to the method of the present invention;

FIG. 8 is a partial, cutaway, lateral perspective view of another embodiment of a drainage device shown in FIG. 1;

FIG. 9 is a partial, lateral perspective view of an alternate embodiment of the occlusion removing structure shown as part of the drainage device shown in FIG. 8;

FIG. 10 through FIG. 12 show various steps in a method of unoccluding an occluded drain according to the present invention, using the drainage device shown in FIG. 8;

FIG. 13 is a lateral perspective view of an introducer with a replacement occlusion removing structure within the introducer according to the present invention;

FIG. 14 is a bottom perspective view of the introducer with a replacement occlusion removing structure within the introducer shown in FIG. 13;

FIG. 15 is a partial, cutaway, lateral perspective view of a device according to the present invention having the replacement occlusion removing structure inserted into the device;

FIG. 16 is another partial, cutaway, lateral perspective view of a device according to the present invention having the replacement occlusion removing structure inserted into the device;

FIG. 17 is a partial, lateral perspective view of an embodiment of an occlusion removing structure for use in a device according to the present invention;

FIG. 18 is a partial, cutaway, lateral perspective view of another embodiment of a drainage device according to the present invention comprising the occlusion removing structure shown in FIG. 17;

FIG. 19 is a partial cutaway, lateral perspective view of a drainage device shown in FIG. 18 connected to a suction device, and with the distal end of the device within a drainage area;

FIG. 20 is a partial cutaway, lateral perspective view of a drainage device shown in FIG. 18 connected to a suction device, showing material occluding the central lumen of the drain;

FIG. 21 is a partial cutaway, lateral perspective view of a drainage device shown in FIG. 18 showing the occluding material being removed from the drain with an occlusion removing structure;

FIG. 22 is a partial, cutaway, lateral perspective view of another embodiment of a drainage device according to the present invention comprising a drain with an occlusion removing structure;

FIG. 23 is another partial, cutaway, lateral perspective view of the embodiment of the device shown in FIG. 22 showing an occlusion being removed from the device according to the method of the present invention; and

FIG. 24 is another partial, cutaway, lateral perspective view of the embodiment of the device shown in FIG. 22 showing an occlusion being removed from the device according to the method of the present invention.

### DESCRIPTION

According to one embodiment of the present invention, there is provided a drainage device comprising an occlusion removing structure. According to another embodiment of the present invention, there is provided a method of unoccluding an occluded drain. In a preferred embodiment, the method comprises providing a device according to the present invention. The device and method prolongs the useful life of a drain, such as a medical drain, and obviate the need for replacing the drain when it becomes occluded, thereby decreasing the cost associated with drain replacement, and the risk of patient morbidity associated with drain replacement. Though the drainage device of the present invention is presented primarily in the context of a medical drain in this disclosure, the drainage device can also be used for other non-surgical purposes, as will be understood by those with skill in the art with reference to this disclosure. The device and method will now be presented in detail.

As used in this disclosure, the term “comprise” and variations of the term, such as “comprising” and “comprises,” are not intended to exclude other additives, components, integers or steps.

As used in this disclosure, the term “occlude” and variations of the term, such as “occluded,” “occluding,” and “occlusion” means a mass or clog of occluding material within the central lumen of a drain, which either partially or completely decreases the function of a drain.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

The devices of the present invention and their component parts comprise any suitable material for the intended purpose of the device, as will be understood by those with skill in the art with reference to this disclosure. For example, when used as a medical drain, the device will usually comprise one or more than one biocompatible material capable of being sterilized, and the drain portion of the device will preferably comprise material that can be easily cut to a desired length as appropriate for the intended use. By way of example only, in one embodiment when the device is used as a medical drain, the drain portion of the device has an outer diameter of between about 3 and about 10 mm (9-30 French).

As will be understood by those with skill in the art with reference to this disclosure, the device of the present invention can be used for a variety of both surgical and non-surgical uses. Examples of surgical uses include biliary tubes and stents, chest tubes, decompression catheters, feeding tubes, gastrointestinal decompression catheters, gastrostomy tubes, jejunostomy tubes, mediastinal tubes, nasogastric tubes, nephrostomy catheters, percutaneous drainage catheters, peritoneal dialysis catheters such as abscess drainage catheters, vascular catheters such as venous hemodialysis catheters, and ventriculostomy tubes. Further, the device of the present invention can be used as biliary and urinary drainage catheters, such as Foley-type bladder catheters placed after bloody surgeries, such as prostate resections, where drain tubes particularly tend to occlude with blood clots. Additionally, the device of the present invention can be utilized as a sump-type tube, that is, a drain possessing an additional lumen used to draw external air into the patient while suction is applied to the drain to prevent adhesion of the drain's side apertures to the surrounding tissues within the wound or body cavity, thereby preventing fluid drainage through the drain. Therefore, as will be understood by those with skill in the art with reference to this disclosure, though the present invention is disclosed in terms of a “drainage device” comprising a “drain,” neither the device nor the method of use are intended to be limited to draining only, but encompass any other use as appropriate for the structure.

In one embodiment, the present invention comprises a drainage device. The drainage device comprises means for drainage and means for removing an occlusion. In each of the embodiments of the drainage device disclosed, the means for drainage and the means for removing an occlusion will be identified or will be understood by those with skill in the art with reference to this disclosure. In general, the means for removing an occlusion is the occlusion removing structure referred to in this disclosure, and the means for drainage is the remainder of the device.

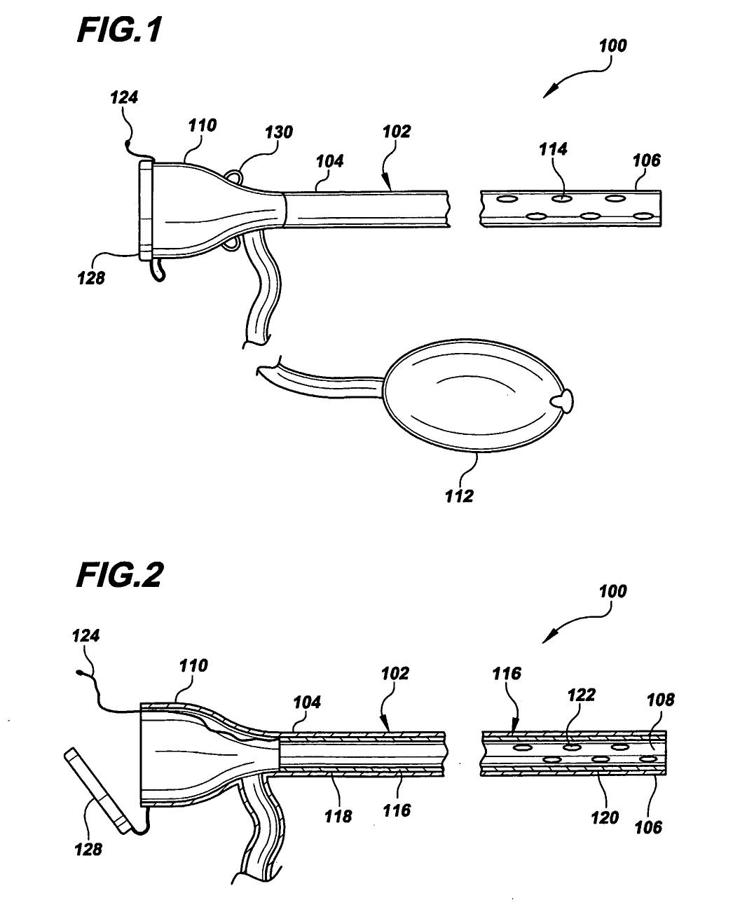

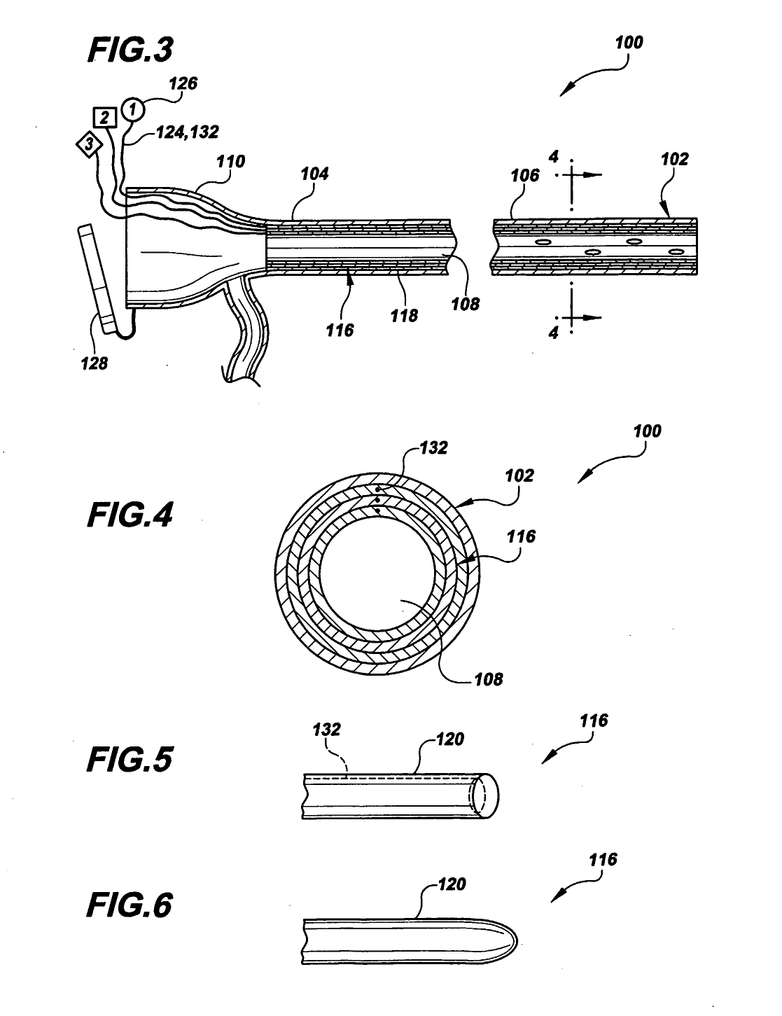

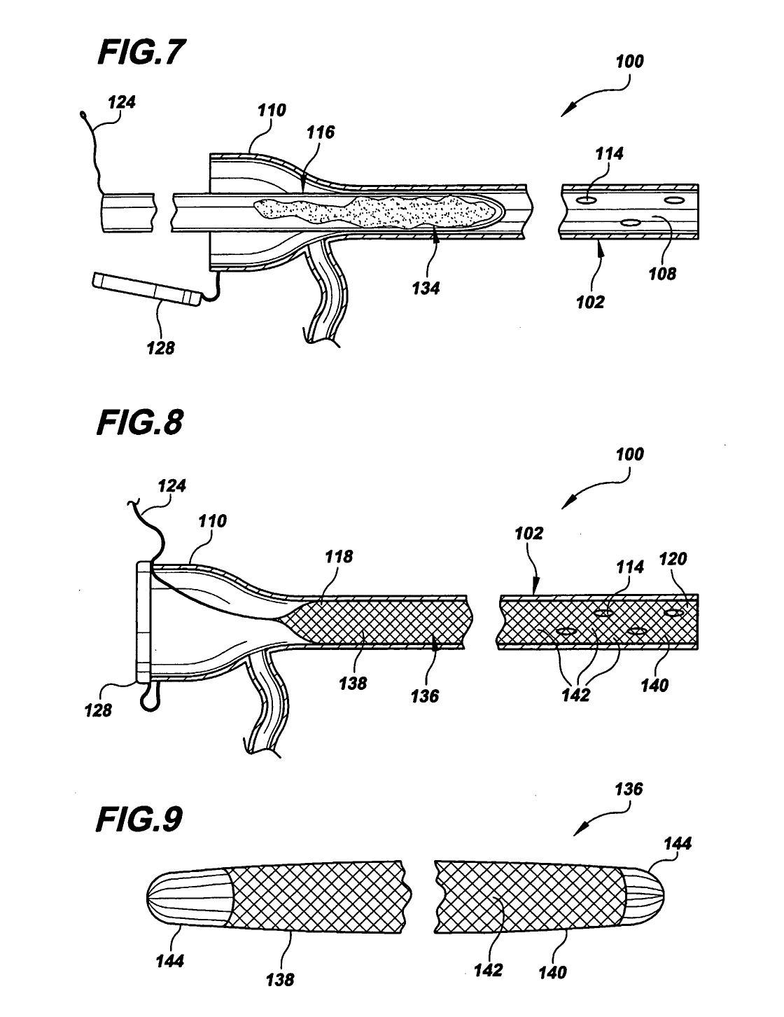

Referring now to FIG. 1 through FIG. 7, there are shown, respectively, a partial, lateral perspective view of one embodiment of a drainage device according to the present invention comprising a drain with an occlusion removing structure, shown with a suction device attached (FIG. 1); a partial, cutaway, lateral perspective view of the embodiment of the device shown in FIG. 1 (FIG. 2); a partial, cutaway, lateral perspective view of another embodiment of a drainage device shown in FIG. 1, with an alternate embodiment of the occlusion removing structure (FIG. 3); a cross-sectional view of the device shown in FIG. 3, taken along line 4-4 (FIG. 4); a close-up view of the distal end of the occlusion removing structure of one embodiment of the drainage device shown in FIG. 1 comprising a snare with the snare open (FIG. 5); another close-up view of the distal end of the occlusion removing structure of one embodiment of the drainage device shown in FIG. 1 comprising a snare with the snare closed (FIG. 6); another partial, cutaway, lateral perspective view of the embodiment of the device shown in FIG. 1 showing an occlusion being removed from the device according to the method of the present invention (FIG. 7).

As can be seen, in one embodiment of the present invention, the drainage device 100 comprises a drain 102 comprising a proximal end 104, a distal end 106 and a central lumen 108. In a preferred embodiment, the proximal end 104 of the drain 102 comprises an adaptor 110 configured to mate with a suction device 112. The adaptor 110 can comprise a side-arm connecting tube, as shown in FIG. 1, or can be configured to mate with a suction device through the proximal-most opening of the adaptor 110, or can be configured to mate with a suction device in another manner, as will be understood by those with skill in the art with reference to this disclosure. In a particularly preferred embodiment, such as when the device 100 is being used as a medical drain, the suction device 112 is a bulb-type suction device as shown in FIG. 1; however, the suction device 112 can be any suitable suction device, as will be understood by those with skill in the art with reference to this disclosure. In a particularly preferred embodiment, the device 100 itself further comprises a suction device 112 configured to mate with the adapter 110. The suction device 112 can be any suitable suction device as will be understood by those with skill in the art with reference to this disclosure, such as for example a bulb-type suction device as shown in FIG. 1.

The distal end 112 of the drain 102 comprises one or more than one aperture 114 extending through the wall of the drain 102 from the central lumen 108 to the exterior of the drain 102. In one embodiment, one of the apertures 114 on the distal end 106 of the drain 102 is at the distal-most end of the distal end 106 of the drain, as shown in FIG. 1, FIG. 2, FIG. 3 and FIG. 7.

The drainage device 100 further comprises an occlusion removing structure 116. The occlusion removing structure 116 comprises one or more than one tubular structure configured to fit within the central lumen 108 of the drain 102. In one embodiment, as shown in FIG. 2, the occlusion removing structure 116 comprises only one tubular structure. In a preferred embodiment, as shown in FIG. 3 and FIG. 4, the occlusion removing structure 116 comprises a plurality of co-axial tubular structures configured to fit within the central lumen 108 of the drain 102. The occlusion removing structure 116 comprises a proximal end 118 and a distal end 120. The distal end 120 of the occlusion removing structure 116 comprises one or more than one aperture 122 extending through the wall of the occlusion removing structure 116. In a preferred embodiment, the apertures 114 and 122 are arranged such that, when the occlusion removing structure 116 is placed within the drain 102, the apertures 114 and 122 align, permitting drainage to enter the central lumen 108 of the drain 102 from the drainage area surrounding the distal end 106 of the drain 102 of the device 100. In a particularly preferred embodiment, the number of apertures 114 on the distal end 106 of the drain 102 equal the number of apertures 122 on the distal end 120 of the occlusion removing structure 116. One of the apertures 122 on the distal end 120 of the occlusion removing structure 116 can be at the distal-most end of the distal end 120 of the occlusion removing structure 116, as shown in FIG. 2, FIG. 3 and FIG. 7.

In a preferred embodiment, as shown in FIG. 1 through FIG. 4, the proximal end 118 of the occlusion removing structure 116 comprises an appendage 124. In the embodiment shown in FIG. 3, when the occlusion removing structure 116 comprises a plurality of co-axial tubular structures, each tubular structure preferably comprises a separate appendage 124. The appendage 124 is configured to permit an operator to remove the occlusion removing structure 116 from of the drain 102, as shown in FIG. 2, or to remove one of the co-axial tubular structures, as shown in FIG. 3, by applying proximal traction on the appendage 124. In one embodiment, as shown, the appendage 124 comprises a long, thin strip of material, with or without an expanded end or loop at the proximal-most end, or comprises a gathering of the material that comprises the occlusion removing structure 116. The appendage 124, when present, is configured to extend proximally through the adaptor 108 when present, or through a valve in the adaptor 108, as shown in FIG. 1, FIG. 2 and FIG. 3. When the occlusion removing structure 116 comprises a plurality of co-axial tubular structures, and each structure preferably comprises a separate appendage 124, each appendage 124 preferably comprises a separate identification tag 126 to assist the operator in selecting the proper appendage 124 attached to each of the plurality of co-axial tubular structures to apply proximal traction to the tubular structure.

In another preferred embodiment, the drain 102 further comprises a cap 128 that prevents contamination through the proximal end 104 of the drain 102 during use. In a particularly preferred embodiment, the cap 128 is a resealable, snap-open type cap as shown in FIG. 1, FIG. 2, FIG. 3 and FIG. 7, and can comprise a valve within the cap 128. The cap 128 can, however, be any structure suitable for the purpose, as will be understood by those with skill in the art with reference to this disclosure, such as for example, a screw-on cover. When the cap 128 is in the closed position, as shown in FIG. 1 and FIG. 3, the cap 128 engages the proximal end of each appendage 124 present preventing movement of the appendage 124, as well as preventing contamination through the proximal end 104 of the drain 102. When the cap 128 is in the open position, as shown in FIG. 2, however, the occlusion removing structure 116 can be translated proximally out of the drain 102, as shown in FIG. 7, by applying proximal traction on the appendage 124. In another preferred embodiment, either the outer surface of the occlusion removing structure 116, or the inner surface of the drain 102, or both have a lubricious coating to aid in separation of the occlusion removing structure 116 from the drain 102 during proximal translation of the occlusion removing structure 116. In another embodiment, the drain 102 comprises one or more than one structure 130 for securing the proximal end 104 of the device 100, such as one or more than one loop, as shown, for securing the proximal end of the device 100 to the skin of a patient, when the device 100 is used as a medical drain, or to another structure, when the device 100 is used as a non-medical drain.

Referring now to FIG. 5 through FIG. 7, in another preferred embodiment, the occlusion removing structure 116 comprises a snare 132 embedded in the wall of the occlusion removing structure 116. When present, applying proximal traction to the proximal end of the snare 132, which can be the appendage 124, causes the distal end 120 of the occlusion removing structure 116 to contract in a purse-string type fashion, thereby substantially or completely sealing the distal end 120 of the occlusion removing structure 116, and preventing any occluding material present in the occlusion removing structure 116 to remain in the occlusion removing structure 116 during removal of the occlusion removing structure 116 from the drain 102. When the occlusion removing structure 116 comprises a plurality of co-axial tubular structures, as shown in FIG. 3 and FIG. 4, each co-axial tubular structure preferably comprises a separate snare 132.

In another embodiment, the present invention comprises a method of unoccluding an occluded drain. The method comprises, first, providing a drainage device 100 according to the present invention, such as the device shown in FIG. 1 through FIG. 7. Next, the distal end 106 of the drain 102 is placed within an area to be drained and allowed to drain drainage for the area. Then, after the central lumen 108 of the drain 102 is occluded with an occluding material 134, the occlusion removing structure 116 is removed from the drain 102 by applying proximal traction to the proximal end 118 of the occlusion removing structure 116, thereby removing the occluding material 134 within the occlusion removing structure 116. In one embodiment, the method comprises attaching the device 100 to a suction device 112 after placing the distal end 112 of the drain 102. In another embodiment, the method comprises reattaching the device 100 to the suction device 112 after removing the occluding material 134 removing structure 116.. As will be understood by those with skill in the art with reference to this disclosure, when the occlusion removing structure 116 comprises an appendage 124, removing the occlusion removing structure 116 comprises applying proximal traction to the appendage 124, thereby translating the occlusion removing structure 116 proximally. Further, as will be understood by those with skill in the art with reference to this disclosure, when the drain 102 comprises a cap 128, removing the occlusion removing structure 116 comprises opening the cap 128 to permit the occlusion removing structure 116 to be removed from the drain 102. Additionally, when the occlusion removing structure 116 comprises a plurality of co-axial tubular structures, as shown in FIG. 3 and FIG. 4, removing the occlusion removing structure 116 preferably comprises removing only one of the plurality of co-axial tubular structures, and the method can optionally comprise infusing a fluid, such as saline, between the separate co-axial tubular structures to facilitate separation of the co-axial structures from each other. Further, when the occlusion removing structure 116 comprises a snare 132, removing the occlusion removing structure 116 comprises applying proximal traction to the proximal end of the snare 132 causing the distal end 120 of the occlusion removing structure 116 to contract in a purse-string type fashion, thereby substantially or completely sealing the distal end of the occlusion removing structure 116, and preventing any occluding material 134 present in the occlusion removing structure 116 to remain in the occlusion removing structure 116 during removal of the occlusion removing structure 116 from the drain 102. When the occlusion removing structure 116 comprises a plurality of co-axial tubular structures, as shown in FIG. 3 and FIG. 4, each co-axial tubular structures comprises a separate snare 132.

Referring now to FIG. 8, there is shown a partial, cutaway, lateral perspective view of another embodiment of a drainage device according to the present invention. As can be seen, the device 100 comprises a drain 102, as disclosed above, and an occlusion removing structure 136. In this embodiment, the occlusion removing structure 136 comprises a proximal end 138 and a distal end 140. The occlusion removing structure 136 is configured to extend substantially throughout the length of the drain 102, with the proximal end 138 of the occlusion removing structure 136 extending through the proximal end of the drain 104, and through the cap 128, when present, as an appendage 124. The occlusion removing structure 136 comprises a tubular mesh and, when used as a medical drain, preferably comprises a self-expanding, flexible, kink-resistant, biocompatible mesh material, such as for example, as ELGILOY®, nitinol, stainless steel, or a biocompatible plastic or shape-memory plastic polymer. In one embodiment, when the device 100 is being used as a medical drain, the occlusion removing structure 136 comprises, for example, between about 12 and about 20 braided wires, each wire having a diameter of between about 0.1 mm and 0.15 mm. In another preferred embodiment, the wires comprise an anti-thrombogenic coating. In another preferred embodiment, the occlusion removing structure 136 comprises material that can be easily cut to a desired size. As will be understood by those with skill in the art with reference to this disclosure, when the occlusion removing structure 136 is in place within the drain 102, as shown in FIG. 8, the plurality of apertures 142 in the mess structure of the occlusion removing structure 136 permits drainage to enter the central lumen 108 of the drain 102 from the drainage area surrounding the distal end 106 of the drain 102 of the device 100. Referring now to FIG. 9, there is shown a partial, lateral perspective view of an alternate embodiment of the occlusion removing structure 136, where the proximal end 138 of the occlusion removing structure 136 comprises a plurality of loops 144.

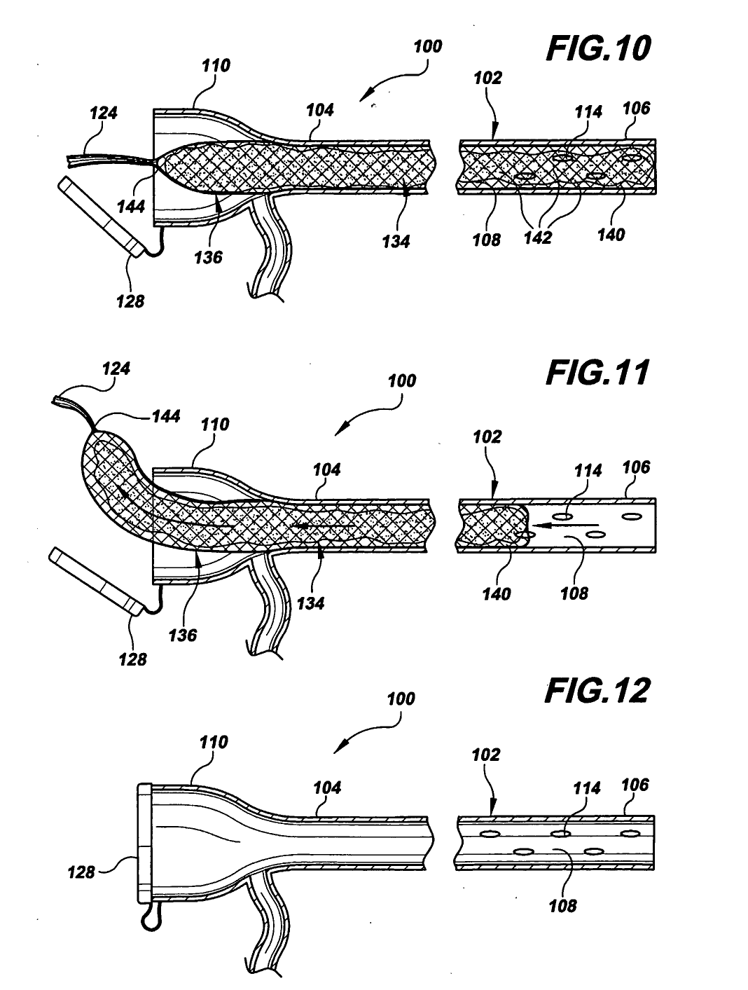

In another embodiment, the present invention comprises a method of unoccluding an occluded drain. Referring now to FIG. 10 through FIG. 12, there are shown various steps in a method of unoccluding an occluded drain according to the present invention, using the drainage device shown in FIG. 8. As can be seen, the method comprises, first, providing a drainage device 100 according to the present invention, such as the device shown in FIG. 8. Then, the distal end 106 of the drain 102 is placed within an area to be drained and allowed to drain drainage from the area. Next, after the central lumen 108 of the drain 102 is occluded with an occluding material 134, as shown in FIG. 10, the occlusion removing structure 136 is removed from the drain 102 by applying proximal traction to the proximal end 118 of the occlusion removing structure 136, as shown in FIG. 11, thereby removing the occluding material 134 within the occlusion removing structure 136 and unoccluding the drain 102. As proximal traction is applied to the occlusion removing structure 136, the distal end 140 elongates, thereby constricting the tubular mesh around any occluding material 134 within the central lumen 108 of the drain 102 and assisting in removing the occluding material 134.

In one embodiment, the method comprises attaching the device 100 to a suction device 112 after placing the distal end 112 of the drain 102. In another embodiment, the method comprises reattaching the device 100 to the suction device 112 after removing the occlusion removing structure 136. As will be understood by those with skill in the art with reference to this disclosure, when the occlusion removing structure 136 comprises an appendage 124, removing the occlusion removing structure 136 can comprise applying proximal traction to the appendage 124, thereby translating the occlusion removing structure 136 proximally. Further, as will be understood by those with skill in the art with reference to this disclosure, when drain 102 comprises a cap 128, removing the occlusion removing structure 136 comprises opening the cap 128 to permit the occlusion removing structure 136 to be removed from the drain 102. In another embodiment of the method, the occlusion removing structure 136 is removed by grasping the loops 144 at the proximal end 138 of the embodiment of the occlusion removing structure 136 shown in FIG. 9 by a hook and pulling the proximal end 138 into a sheath (not shown).

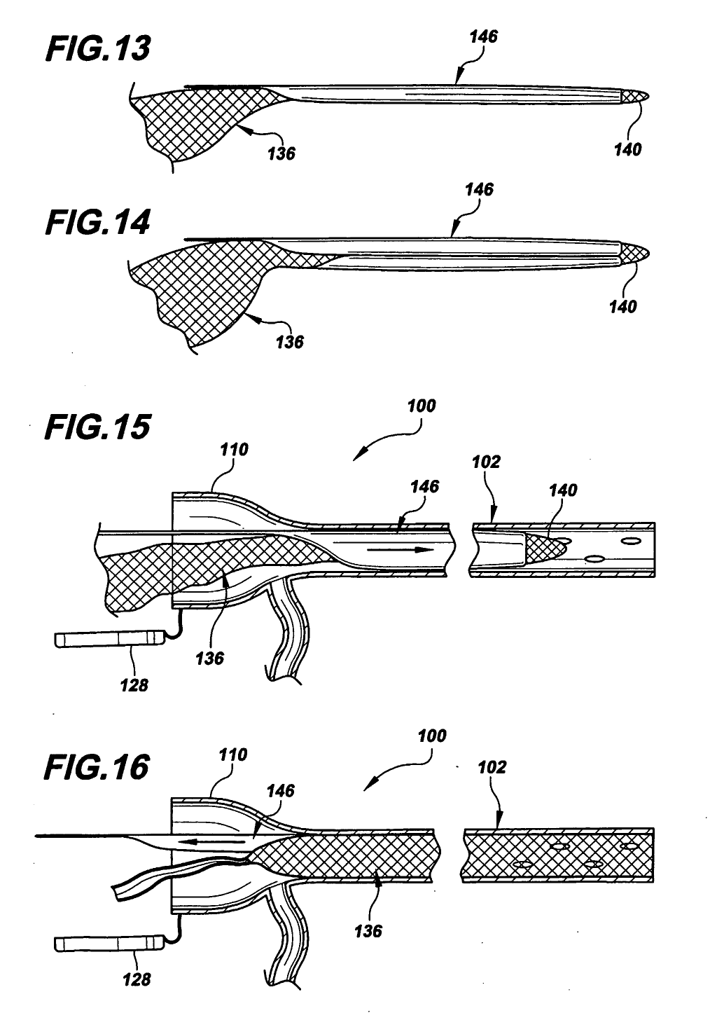

In a preferred embodiment, the method comprises replacing the occlusion removing structure 136 in the drain 102 after unoccluding the drain 102. Referring now to FIG. 13 through FIG. 16, there are shown various structures and steps used in replacing the occlusion removing structure 136 in the drain 102 after unoccluding the drain 102, where FIG. 13 shows a lateral perspective view of an introducer 146 with a replacement occlusion removing structure 136 within the introducer 146; FIG. 14 shows a bottom perspective view of an introducer 146 with a replacement occlusion removing structure 136 within the introducer 146; FIG. 15 shows a partial, cutaway, lateral perspective view of a device 100 having the replacement occlusion removing structure 136 inserted into the device 100; and FIG. 16 shows another partial, cutaway, lateral perspective view of a device 100 having the replacement occlusion removing structure 136 inserted into the device 100. As can be seen, in one embodiment, the replacement occlusion removing structure 136 is contained in an introducer 146, such as a metal or plastic tube, as shown in FIG. 13 and FIG. 14. The step of replacing the occlusion removing structure 136 in the drain 102 after unoccluding the drain 102 comprises advancing the introducer 146 containing the replacement occlusion removing structure 136 through the proximal end 104 of the unoccluded drain 102 until the distal end 140 of the replacement occlusion removing structure 136 is placed at or near the distal end 106 of the drain 102. Then, the introducer 146 is removed from the drain 102, leaving the replacement occlusion removing structure 136 within the drain 102. Next, the drain 102 is reattached to a suction device 112 as necessary. In a preferred embodiment, the introducer 146 comprises flexible material to allow the introducer 146 containing the replacement occlusion removing structure 136 to negotiate bends or curves in the drain 102. In another preferred embodiment, the distal end 148 of the introducer 146 is blunted to prevent the distal end 148 of the introducer 146 from becoming impacted in apertures 114 in the distal end 106 of the drain 102.

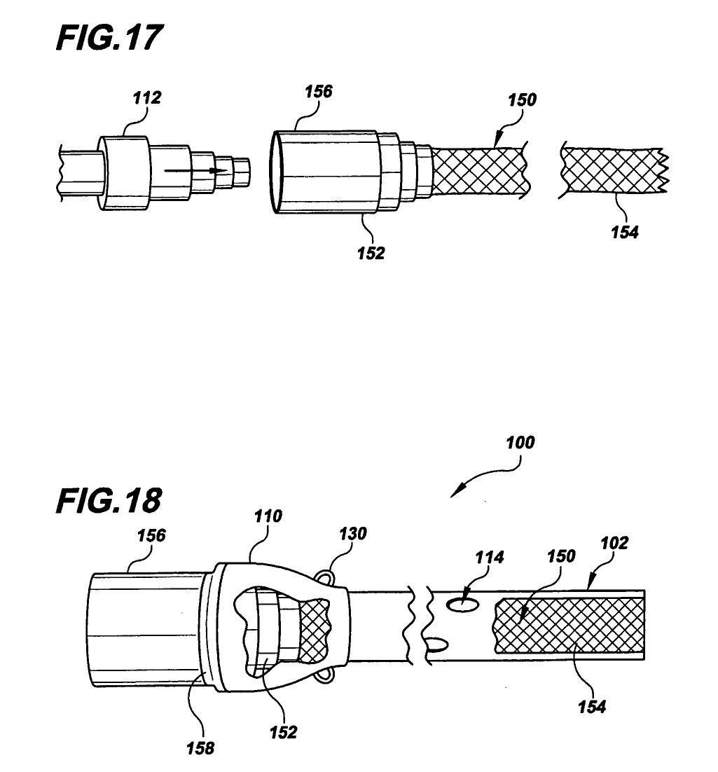

Referring now to FIG. 17 and FIG. 18, there is shown a partial, lateral perspective view of an embodiment of an occlusion removing structure for use in a device according to the present invention (FIG. 17); and a partial, cutaway, lateral perspective view of another embodiment of a drainage device according to the present invention comprising the occlusion removing structure shown in FIG. 17 (FIG. 18). As can be seen, the occlusion removing structure 150 comprises a proximal end 152 and a distal end 154. The distal end 154 corresponds to the distal end 140 of the occlusion removing structure 136, disclosed in this disclosure. The proximal end 152, however, comprises a connector 156. In a preferred embodiment, as shown in FIG. 17 and FIG. 18, the connector 156 is a stepped, male connector, (a “Christmas Tree” type connector) capable of mating to a plurality of sizes of female connectors. In a preferred embodiment, the adaptor 110 of the drain 102 is configured to mate with the connector 156, as can be seen in FIG. 18. In another preferred embodiment, the device 100 further comprises a sealant 158 for sealing the joint between the connector 156 and the adaptor 110, such as for example, an adhesive strip as shown in FIG. 18.

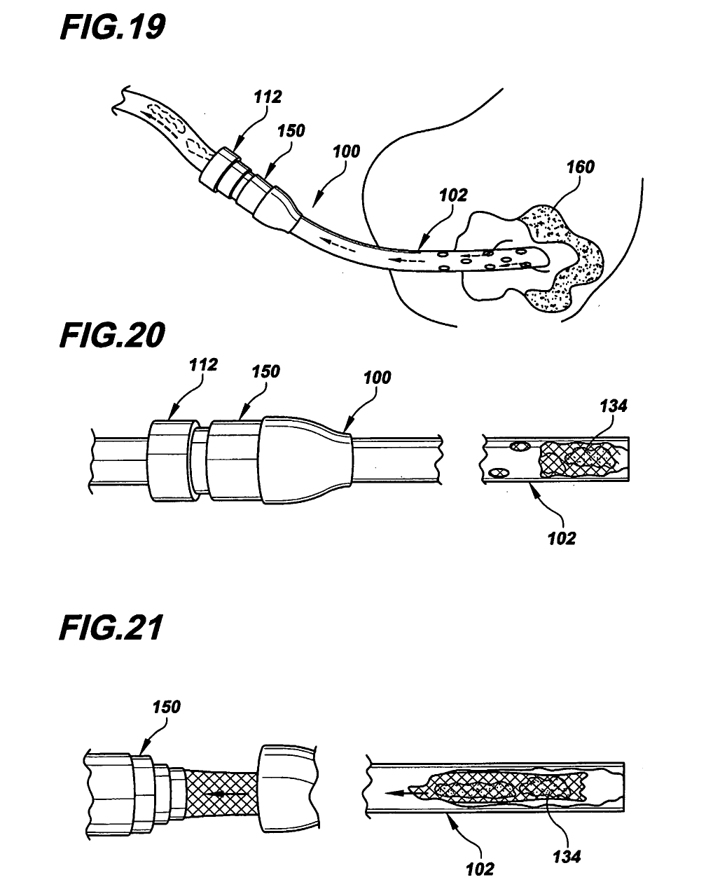

In another embodiment, the present invention comprises a method of unoccluding an occluded drain. Referring now to FIG. 19 through FIG. 21, there are shown various steps in a method of unoccluding an occluded drain according to the present invention, using the drainage device shown in FIG. 18, where FIG. 19 is a partial cutaway, lateral perspective view of a drainage device shown in FIG. 18 connected to a suction device, and with the distal end of the device within a drainage area; FIG. 20 is a partial cutaway, lateral perspective view of a drainage device shown in FIG. 18 connected to a suction device, showing material occluding the central lumen of the drain; and FIG. 21 is a partial cutaway, lateral perspective view of a drainage device shown in FIG. 18 showing the occluding material being removed from the drain with the occlusion removing structure. As can be seen, the method comprises, first, providing a drainage device 100 according to the present invention, such as the device shown in FIG. 18. Then, the distal end 106 of the drain 102 is placed within an area to be drained 160 and allowed to drain drainage for the area, as shown in FIG. 19. Next, after the central lumen 108 of the drain 102 is occluded with occluding material 134, as shown in FIG. 20, the occlusion removing structure 150 is removed from the drain 102 by, first, disconnecting the connector 156 from the adaptor 110, and then applying proximal traction to the proximal end 152 of the occlusion removing structure 150, as shown in FIG. 21, thereby removing the occluding material 134 within the occlusion removing structure 150 and unoccluding the drain 102. As proximal traction is applied to the occlusion removing structure 150, the mesh at the distal end 154 elongates, thereby constricting the tubular mesh around any occluding material 134 within the central lumen 108 of the drain 102 and assisting in removing the occluding material 134. In one embodiment, the method comprises attaching the device 100 to a suction device 112 after placing the distal end 112 of the drain 102. In another embodiment, the method comprises reattaching the device 100 to the suction device 112 after removing the occlusion removing structure 150.

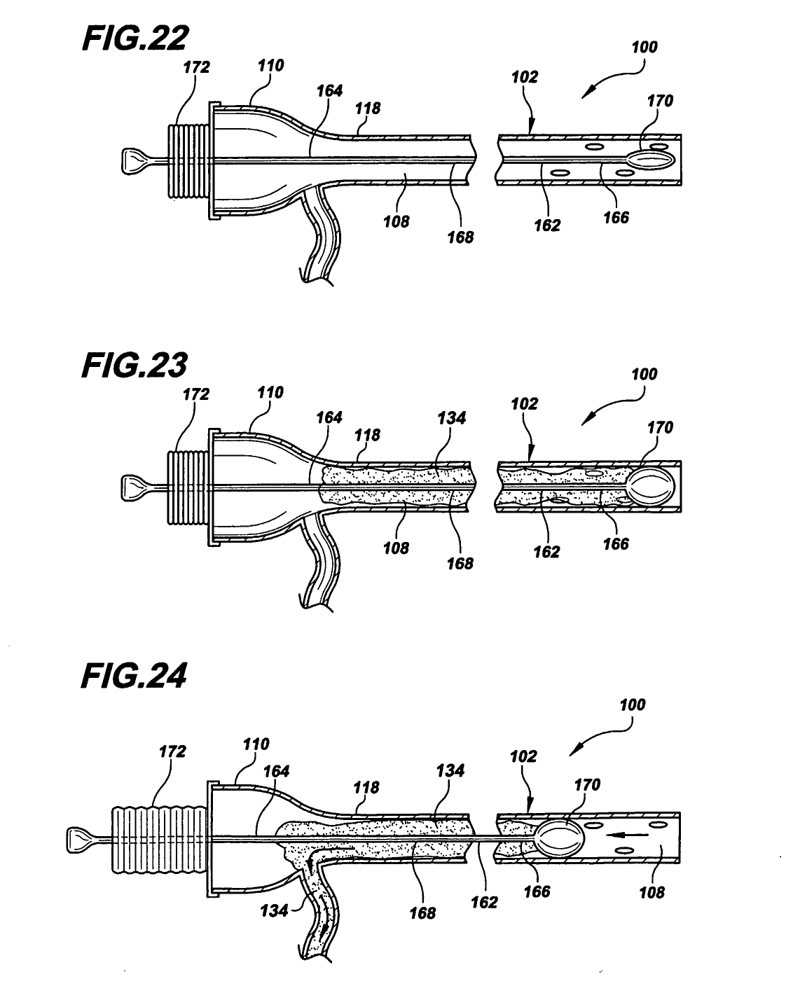

Referring now to FIG. 22 through FIG. 24, there are shown, respectively, a partial, cutaway, lateral perspective view of another embodiment of a drainage device according to the present invention comprising a drain with an occlusion removing structure (FIG. 22); another partial, cutaway, lateral perspective view of the embodiment of the device shown in FIG. 22 showing an occlusion being removed from the device according to the method of the present invention (FIG. 23); and another partial, cutaway, lateral perspective view of the embodiment of the device shown in FIG. 22 showing an occlusion being removed from the device according to the method of the present invention (FIG. 24). As can be seen, in another embodiment of the present invention, the drainage device 100 comprises a drain 102 and an occlusion removing structure 162 comprising a proximal end 164, a distal end 166, and an intermediate segment 168 between the proximal end 164 and the distal end 166. The distal end 166 comprises an inflatable balloon 170. The proximal end 164 is an injection hub configured to mate with a source of inflation material, not shown, for example, an inflation fluid, such as saline, or an inflation gas. The intermediate segment 168 comprises an inflation lumen extending from the proximal end 164 to the inflatable balloon 170 at the distal end 166. In a preferred embodiment, the proximal end 104 of the drain 102 further comprises a collapsible sleeve 172, as shown in FIG. 22 through FIG. 24. In another preferred embodiment, the drain 102 can also comprise a cap 128, not shown, on the proximal end 104 as disclosed in connection with other embodiments of the device 100.

In another embodiment, the present invention comprises a method of unoccluding an occluded drain. Referring now to FIG. 22 through FIG. 24, the method comprises, first, providing a drainage device 100 according to the present invention, such as the device shown in FIG. 22 through FIG. 24. Next, the distal end 106 of the drain 102 is placed within an area to be drained and allowed to drain drainage for the area with the inflatable balloon 170 initially in the uninflated state as shown in FIG. 22. Then, after the central lumen 108 of the drain 102 is occluded with occluding material 134, as shown in FIG. 22, the inflatable balloon 170 is inflated by injecting inflation material into the proximal end 164 of the occlusion removing structure 162 and through the intermediate segment 168 to the inflatable balloon 170. Next, the occlusion removing structure 162 is removed from the drain 102 by proximally translating the occlusion removing structure 162, thereby removing the occluding material 134. The occluding material 134 is either removed from the proximal end 104 of the drain 102 or through the suction device 112, as shown in FIG. 24.

In one embodiment, the method further comprises replacing the occlusion removing structure 162 after the occlusion is removed. In another embodiment, the method comprises attaching the device 100 to a suction device 124 after placing the distal end 106 of the drain 102. In another embodiment, the method comprises reattaching the device 100 to the suction device 112 after removing the occlusion removing structure 162. In a preferred embodiment, the drain 102 comprises a collapsible sleeve 172, and the collapsible sleeve 172 is extended proximally as the occlusion removing structure 162 is removed from the drain 102 to maintain sterility of the occlusion removing structure 162, the collapsible sleeve 172 is collapsed as the occlusion removing structure 162 is reinserted into the drain 102. As will be understood by those with skill in the art with reference to this disclosure, when drain the 102 comprises a cap 128, removing the occlusion removing structure 162 comprises opening the cap 128 to permit the occlusion removing structure 162 to be removed from the drain 102.

In another embodiment of the present invention, there is provided a drainage device 100 comprising a plurality of different occlusion removing structures as disclosed in the various embodiments of the device 100. In such an embodiment, the drain 102 is unoccluded using either one of the occlusion removing structures or using a plurality of the occlusion removing structures. The drainage device can comprise any of the occlusion removing structures disclosed in this disclosure in combination, as will be understood by those with skill in the art with reference to this disclosure. For example, the device 100 can comprise an occlusion removing structure 116 comprising one or more than one tubular structure with an occlusion removing structure 162 comprising an inflatable balloon. In a preferred embodiment, the device 100 comprises an occlusion removing structure 116 comprising a plurality of co-axial tubular structures with an occlusion removing structure 162 comprising an inflatable balloon between each of the plurality of co-axial tubular structures. This embodiment is particularly suitable for use as in-dwelling feeding and infusion tubes, where the inflatable balloon 170 is kept inflated between periods of infusion. In another preferred embodiment, the device 100 comprises a tubular mesh occlusion removing structure 136, 142 with an occlusion removing structure 162 comprising an inflatable balloon.

Although the present invention has been discussed in considerable detail with reference to certain preferred embodiments, other embodiments are possible. Therefore, the scope of the appended claims should not be limited to the description of preferred embodiments contained in this disclosure.

1. A drainage device comprising means for drainage and means for removing an occlusion.

2. A method of unoccluding a means for drainage comprising:
a) providing the drainage device of claim 1;
b) placing the drainage device into an area to be drained;
c) allowing occluding material to occlude the means for drainage; and
d) removing the means for removing an occlusion from the means for drainage, thereby unoccluding the means for drainage.

3. A drainage device comprising:
a) a drain comprising a proximal end, a distal end and a central lumen; and
b) an occlusion removing structure within the central lumen comprising a proximal end and a distal end;
where the proximal end of the drain comprises an adaptor configured to mate with a suction device;
where the distal end of the drain comprises one or more than one aperture;
where the occlusion removing structure comprises one or more than one tubular structure comprising a wall; and
where the distal end of the occlusion removing structure comprises one or more than one aperture.

4. The drainage device of claim 3, further comprising a suction device mated with the adapter.

5. The drainage device of claim 3, where the occlusion removing structure comprises a plurality of co-axial tubular structures.

6. The drainage device of claim 3, where the number of the one or more than one aperture on the distal end of the drain is equal to the number of the one or more than one aperture on the distal end of the occlusion removing structure.

7. The drainage device of claim 3, where the proximal end of the occlusion removing structure comprises an appendage.

8. The drainage device of claim 7, where the appendage comprises an identification tag.

9. The drainage device of claim 3, where the proximal end of the drain comprises a cap.

10. The drainage device of claim 3, where the occlusion removing structure comprises an outer surface, the drain comprises an inner surface, and where the outer surface of the occlusion removing structure, or the inner surface of the drain, or both have a lubricious coating.

11. The drainage device of claim 3, where the drain further comprises one or more than one structure for securing the proximal end of the device.

12. The drainage device of claim 3, where the wall comprises a snare.

13. The drainage device of claim 3, where the occlusion removing structure is a first occlusion removing structure, and where the device further comprises a second occlusion removing structure comprising a proximal end, a distal end, an intermediate segment between the proximal end and the distal end, and an inflation balloon on the distal end.

14. A method of unoccluding a drainage device comprising:
a) providing the drainage device of claim 3;
b) placing the distal end of the drain into an area to be drained;
c) allowing occluding material to occlude the drain; and
d) removing the occlusion removing structure from the drain, thereby unoccluding the drain.

15. The method of claim 14, further comprising attaching the drainage device to a suction device after placing the distal end of the drain.

16. The method of claim 14, further comprising reattaching the drainage device to the suction device after removing the occluding material removing structure.

17. The method of claim 14, where the occlusion removing structure comprises an appendage, and where removing the occlusion removing structure comprises applying proximal traction-to the appendage, thereby translating the occlusion removing structure proximally.

18. The method of claim 14, where the drain comprises a cap, and where removing the occlusion removing structure comprises opening the cap to permit the occlusion removing structure to be removed from the drain.

19. The method of claim 14, where the occlusion removing structure comprises a plurality of co-axial tubular structures, and where removing the occlusion removing structure preferably comprises removing only one of the plurality of co-axial tubular structures.

20. The method of claim 19, further comprising infusing a fluid between each of the plurality of co-axial tubular structures to facilitate separation of the co-axial structures from each other.

21. The method of claim 14, where the wall comprises a snare, and where removing the occlusion removing structure comprises applying proximal traction to the proximal end of the snare causes the distal end of the occlusion removing structure to contract in a purse-string type fashion, thereby substantially or completely sealing the distal end of the occlusion removing structure.

22. A drainage device comprising:
a) a drain comprising a proximal end, a distal end and a central lumen; and
b) an occlusion removing structure within the central lumen comprising a proximal end and a distal end;
where the proximal end of the drain comprises an adaptor configured to mate with a suction device;
where the distal end of the drain comprises one or more than one aperture; and
where the occlusion removing structure comprises a tubular mesh.

23. The drainage device of claim 22, where the proximal end of the occlusion removing structure comprises a plurality of loops.

24. The drainage device of claim 22, further comprising a suction device mated with the adapter.

25. The drainage device of claim 22, where the proximal end of the occlusion removing structure comprises an appendage.

26. The drainage device of claim 22, where the proximal end of the drain comprises a cap.

27. The drainage device of claim 22, where the drain further comprises one or more than one structure for securing the proximal end of the device.

28. The drainage device of claim 22, where the occlusion removing structure comprises a connector on the proximal end.

29. The drainage device of claim 27, where the device further comprises a joint between the adaptor and the connector, and further comprising a sealant for sealing the joint between the connector and the adaptor.

30. The drainage device of claim 22, where the occlusion removing structure is a first occlusion removing structure, and where the device further comprises a second occlusion removing structure comprising a proximal end, a distal end, an intermediate segment between the proximal end and the distal end, and an inflation balloon on the distal end.

31. A method of unoccluding a drainage device comprising:
a) providing the drainage device of claim 22;
b) placing the distal end of the drain into an area to be drained;
c) allowing occluding material to occlude the drain; and
d) removing the occlusion removing structure from the drain, thereby unoccluding the drain.

32. The method of claim 31, further comprising attaching the drainage device to a suction device after placing the distal end of the drain.

33. The method of claim 31, further comprising reattaching the drainage device to the suction device after removing the occluding material removing structure.

34. The method of claim 31, where the occlusion removing structure comprises an appendage, and where removing the occlusion removing structure comprises applying proximal traction to the appendage, thereby translating the occlusion removing structure proximally.

35. The method of claim 31, where the drain comprises a cap, and where removing the occlusion removing structure comprises opening the cap to permit the occlusion removing structure to be removed from the drain.

36. The method of claim 31, where the proximal end of the occlusion removing structure comprises loops, and where removing the occlusion removing structure from the drain comprises pulling the proximal end of the occlusion removing structure into a sheath.

37. The method of claim 31, further comprising replacing the occlusion removing structure in the drain after unoccluding the drain.

38. The method of claim 36, where replacing the occlusion removing structure in the drain after unoccluding the drain comprises advancing an introducer containing the replacement occlusion removing structure through the proximal end of the drain until the distal end of the replacement occlusion removing structure is placed at or near the distal end of the drain, and then removing the introducer leaving the replacement occlusion removing structure within the drain.

39. A drainage device comprising:
a) a drain comprising a proximal end, a distal end and a central lumen; and
b) an occlusion removing structure within the central lumen comprising a proximal end, a distal end and an intermediate segment between the proximal end and the distal end;
where the proximal end of the drain comprises an adaptor configured to mate with a suction device;
where the distal end of the drain comprises one or more than one aperture; and
where the occlusion removing structure comprises an inflation balloon on the distal end.

40. The drainage device of claim 39, where the drain further comprises a suction device mated with the adapter.

41. The drainage device of claim 39, where the proximal end of the drain comprises a cap.

42. The drainage device of claim 39, where the drain further comprises one or more than one structure for securing the proximal end of the device.

43. The drainage device of claim 39, where the drain further comprises a collapsible sleeve.

44. A method of unoccluding a drainage device comprising:
a) providing the drainage device of claim 39;
b) placing the distal end of the drain into an area to be drained;
c) allowing occluding material to occlude the drain;
d) inflating the inflatable balloon; and
e) removing the occlusion removing structure from the drain, thereby unoccluding the drain.

45. The method of claim 44, further comprising attaching the drainage device to a suction device after placing the distal end of the drain.

46. The method of claim 44, further comprising reattaching the drainage device to the suction device after removing the occluding material removing structure.

47. The method of claim 44, where the drain comprises a cap, and where removing the occlusion removing structure comprises opening the cap to permit the occlusion removing structure to be removed from the drain.

48. The method of claim 44, further comprising replacing the occlusion removing structure in the drain after unoccluding the drain.

49. The method of claim 44, where the drain further comprises a collapsible sleeve, and where the collapsible sleeve is extended proximally as the occlusion removing structure is removed from the drain.
